(19) United States
(12) Patent Application Publication
(10) Pub. No.: US 2010/0260812 A1
(43) Pub. Date: Oct. 14, 2010

(54) THICKENED FLUID COMPOSITION COMPRISING CHLORINE DIOXIDE

(75) Inventors: Linda Hratko, Woodbridge Township, NJ (US); Frank Joseph Calabro, Staten Island, NY (US); Barry K. Speronello, Belle Mead, NJ (US)

Correspondence Address: BASF CATALYSTS LLC, 100 CAMPUS DRIVE, FLORHAM PARK, NJ, 07932, US

(73) Assignee: BASF Corporation, Florham Park, NJ (US)

(21) Appl. No.: 12/824,830

(22) Filed: Jun. 28, 2010

Related U.S. Application Data
(63) Continuation of application No. 11/340,248, filed on Jan. 26, 2006, now abandoned.

(51) Int. Cl. A61K 8/20; A61K 33/20; A61K 9/10; A61Q 11/00; A61K 9/00; A61P 17/02

(52) U.S. Cl. 424/401; 424/53; 424/663; 424/400

(57) ABSTRACT

This invention relates to a stable composition and method of making a thickened fluid composition comprising chlorine dioxide. The stable composition includes a mixture containing a chlorite, an acid source, and a thickener component. At least one of the chlorite, the acid source and the thickener component is in particulate form. In an embodiment, the mixture may be a unitary solid body wherein chlorine dioxide is generated upon interaction with an aqueous medium, such as water. A free halogen source may also be added to the mixture in some cases.

### FIELD OF THE INVENTION

This invention relates to chlorine dioxide compositions. In particular the invention relates to a thickened chlorine dioxide composition and a method of preparing the composition.

### BACKGROUND OF THE INVENTION

Chlorine dioxide in low concentrations (i.e. up to 1,000 ppm) has long been recognized as useful for the treatment of odors and microbes, see U.S. Pat. No. 6,238,643. Its use is particularly advantageous where microbes and/or organic odorants are sought to be controlled on and around foodstuffs, as chlorine dioxide functions without the formation of undesirable side products such as chloramines or chlorinated organic compounds that can be produced when elemental chlorine is utilized for the same or similar purposes. For example, if a low concentration of chlorine dioxide gas can be maintained in contact with fresh produce for several days during shipping from the farm to the local retailer, the rate of spoilage of the produce can be decreased. In addition, chlorine dioxide gas is also generally considered to be safe for human contact at the low concentrations that are effective for deodorization and most antimicrobial applications.

Further uses of chlorine dioxide are exemplified in the patents disclosed herein below as well as the methods for forming chlorine dioxide. U.S. Pat. No. 2,071,091 discloses an improved fungicide and bactericide, and an improved sterilization process using chlorous acid and the salts of chlorous acid. The term “chlorous acid and the salts of chlorous acid” includes aqueous solutions of soluble chlorite salts that have been acidified to an acidic pH. Such solutions contain mixtures of chlorine dioxide and chlorite anions with the ratio of chlorine dioxide to chlorite being higher when the pH of the solution is lower. This process requires a relatively high degree of user skill to handle and measure the alkaline chlorite and acid. The requirement for an acidic pH limits the utility of this process when the preferred solution pH is alkaline, and the resultant solution is contaminated with sodium chloride and the solution byproducts of the acid.

U.S. Pat. No. 2,071,094 discloses deodorizing compositions in the form of dry briquettes comprising a dry mixture of a soluble chlorite, an acidifying agent, and a filler of lower solubility. Generation of chlorine dioxide begins as the briquette dissolves in water. This process is suitable for unskilled users, but still requires that the resultant solution be produced at an acidic pH, and it is still contaminated with the solution byproducts of the reagents. Furthermore, the inert, low solubility filler leaves an insoluble residue paste that is difficult to handle and dispose of.

U.S. Pat. No. 4,585,482 discloses a long-acting biocidal composition comprising a chlorine dioxide liberating compound and a hydrolyzable organic acid-generating polymer. Methods are disclosed for producing dry polymer encapsulated microcapsules containing such compositions and water such that the resultant dry materials release chlorine dioxide gas. The primary purpose of the polymer encapsulating film of the '482 Patent is to provide for hard, free flowing particles, and to protect against the loss of water from the interior of the microcapsule. Immersing the microcapsules in water would produce a chlorine dioxide solution.

Besides being used to treat odors and microbes, chlorine dioxide may also be used in oral care preparations, teat dips and wound dressings. U.S. Pat. Nos. 5,944,528 and 6,479,037 discloses a tooth whitening composition including a first formulation having a chlorine dioxide precursor and a second formulation having an acidulant capable of generating chlorine dioxide upon contact with the precursor. In one embodiment, the two formulated portions may be mixed thoroughly prior to placing the entire admixed composition into a custom fabricated ethylene vinyl acetate dental tray for application to the teeth. Alternatively, one of the first and second formulations may initially be applied to the teeth prior to the application of the remaining formulation.

U.S. Pat. No. 4,330,531 discloses a germ-killing material and applicator for dispensing germ-killing compositions containing chlorine dioxide. U.S. Pat. No. 5,200,171 discloses an oral health preparation and method. The '171 patent describes a stable mouth wash or dentifrice composition containing stabilized chlorine dioxide and phosphates, the phosphates being present in a range between about 0.02%-3.0%. The stabilized chlorine dioxide is formed using an activating inhibitor, the phosphates, to lower the pH at the time the oral preparation is used in the mouth.

U.S. Pat. No. 6,312,670 discloses tooth bleaching compositions having hydrogen peroxide-containing compounds and methods for bleaching teeth. The composition is capable of administration by means of a dental tray.

U.S. Pat. No. 6,500,408 discloses an enamel-safe tooth bleach and method for use. The dental bleach includes a bleaching agent and a thickening agent. The bleaching agent is typically a peroxide and the thickening agent is polyvinylpyrrolidone. The bleaching may take place using a dental tray. Bleach may be placed against a flexible strip which is placed onto the teeth to be bleached.

U.S. Pat. No. 6,379,685 discloses acidic aqueous chlorite teat dip with improved emollient providing shelf life, sanitizing capacity and tissue protection. The composition can be mixed using two parts, a simple chlorite solution and an acid.

U.S. Pat. No. 5,597,561 discloses adherent disinfecting compositions and methods of use in skin disinfection. The disinfecting composition is directed to the prevention of microbial infections and comprise a protic acid, a metal chlorite and a gelling agent which, when combined, provide an effective adherent matrix that acts as a disinfectant barrier for preventing transmission and propagation of microbial infections.

In addition to the uses and methods cited above, the present assignee has also developed and patented a method of generating chlorine dioxide. The present assignee manufactures Aspetrol® chlorine dioxide generating tablets disclosed in U.S. Pat. Nos. 6,699,404 and 6,432,322. The tablets are used in a wide array of applications such as to oxidize foul smelling compounds, deodorize areas, disinfect, treat and/or purify water, etc. These patents disclose solid bodies for preparing highly converted solutions of chlorine dioxide when added to water. The solid body comprises a metal chlorite such as sodium chlorite, an acid source such as sodium bisulfate and optionally a source of free halogen such as the sodium salt of dichloroisocyanuric acid or a hydrate thereof.

U.S. Pat. No. 6,238,643, also issued to the present assignee, discloses a method of producing an aqueous solution of chlorine dioxide from the reaction of chlorine dioxide generating components. The chlorine dioxide generating components are a metal chlorite and an acid forming component which do not react to produce chlorine dioxide in the substantial absence of water. The chlorine dioxide generating components are disposed in a membrane that is water and/or water vapor permeable but impermeable to the chlorine dioxide generating components contained therein. The membrane containing the chlorine dioxide generating components are immersed in a liquid so the chlorine dioxide may generate and pass out through the membrane into the liquid forming the aqueous solution of chlorine dioxide.

The above-cited patents disclose uses and methods for forming chlorine dioxide solutions. Despite being effective for many different purposes, the unthickened, runny and liquid consistency of many of these solutions limit the potential uses of the solution and often require concerted effort from a user to ensure the solution is being applied in an effective manner. For instance, in tooth whitening applications the majority of professionally-monitored at-home tooth-whitening compositions act by oxidation. These compositions are dispensed into a custom-made tooth-whitening tray for use directly by a patient. Typically, these trays must be held in the mouth of the patient for a period of time often greater than about 60 minutes, and sometimes as long as 8 to 12 hours in order to produce any results.

Furthermore, the limitations of using unthickened, runny chlorine dioxide solutions are apparent when the solution is used in cleaning, sanitizing or disinfecting a surface or substrate, e.g. medical instruments. For example, some methods of applying the chlorine dioxide solution to medical instruments require that the instrument be immersed in the solution. This method of application requires a large amount of the solution to be expended in order to be effective on the instrument. The solution may also be used as a spray to clean, sanitize or disinfect a substrate or area. However, this method of application also presents the problem where the liquid solution could splatter or drip on unintended areas and be ineffective on the desired area. The spraying of unthickened, gaseous or liquid chlorine dioxide may also be insubstantial and require the user to make repeated spray-applications.

The problems in the art such as the runny consistency of the solution and the concerted effort required from the user to apply the solution to a substrate or surface can be overcome by using thickened chlorine dioxide solutions.

Thickened mixtures of chlorine dioxide are as well known in the art as are the aqueous solutions of chlorine dioxide. The thickened mixtures of chlorine dioxide are produced by adding thickener agents such as clays, polymers, gums, etc. to aqueous solutions of chlorine dioxide to produce the thickened and pseudo plastic aqueous fluid mixtures. The advantage of the thickened mixtures comprising chlorine dioxide is the better adherence to vertical surfaces and reduced volatility of chlorine dioxide relative to the unthickened chlorine dioxide solution. The volatility of chlorine dioxide is reduced because the mass transfer of chlorine dioxide from the interior of the thickened mixture to the surface is inhibited.

There is a need to generate chlorine dioxide at high concentrations. Generating such high concentrations of chlorine dioxide has been difficult to produce. Many methods have been used in the art to produce different forms of chlorine dioxide. One method of manufacturing a viscous chlorine dioxide mixture has been to produce a thickened aqueous solution of sodium chlorite and a second thickened aqueous acidic solution and combine the two thickened solutions in the correct ratio at the time of use. Another method involves producing, at the point of use, an aqueous solution of chlorine dioxide using any of the means known in the art (chlorine dioxide generation equipment, mixing solutions of sodium chlorite and acid, mixing solutions of sodium chlorite, acid, and halogen source, etc.) and then adding one or more thickener agents to the chlorine dioxide solution. In all cases, however, the user is required to measure and mix relatively concentrated solutions of sodium chlorite and acid in the field at the point of use, and this requires a relatively high degree of training and skill.

A thickened chlorine dioxide mixture is desired that will have the consistency needed to remain on a surface or substrate for any period of time and be effective thereon without requiring much concerted effort from the user. The present invention provides a stable composition and method of making a thickened mixture comprising chlorine dioxide at high concentrations. The new composition and method provides a way of combining particulate constituent(s) with an aqueous medium to produce the concentrated viscous chlorine dioxide mixture. This invention provides high yield thickened mixtures of chlorine dioxide and overcomes shortcomings of the prior art.

### SUMMARY OF THE INVENTION

This invention relates to a stable composition for forming chlorine dioxide. The composition includes a mixture containing chlorine dioxide forming ingredients such as a chlorite, an acid source, and a thickener component, and optionally water, wherein the ingredients are combined so as to be non-reactive. In one embodiment at least one of the chlorite, the acid source and the thickener component is in particulate form. In another embodiment of this invention, the composition may be a unitary anhydrous body wherein chlorine dioxide is generated upon interaction with water. Alternatively, one or more components of the mixture can be present in an aqueous medium, such as water, as long as the components are in a non-reactive state; for example, the reactive components are treated with a stabilizing component to prevent immediate reaction in water.

### DETAILED DESCRIPTION OF THE INVENTION

The present invention is directed to a stable chlorine dioxide forming composition and method of making a thickened fluid composition comprising chlorine dioxide. The present invention departs from the chlorine dioxide forms of the prior art, which may be unthickened and runny or gaseous. The prior art forms of chlorine dioxide have limited applications due to its consistency. The consistency of the prior art forms of chlorine dioxide often requires a user to make a concerted effort to ensure that the particular type of chlorine dioxide form is maintained on an intended surface. The thickened chlorine dioxide of the present invention, on the other hand, provides better adherence to many substrates and surfaces than unthickened chlorine dioxide solutions. Vertical surfaces are better served by the thickened chlorine dioxide whether used alone or with some sort of chlorine dioxide support device. The thickened chlorine dioxide can exhibit reduced volatility of chlorine dioxide relative to unthickened chlorine dioxide solutions.

The term “stable”, as use herein, is intended to mean that the components used to form chlorine dioxide, i.e., the chlorine dioxide forming ingredients, are not immediately reactive with each other to form chlorine dioxide. In any event the components/ingredients may be combined in any fashion, such as sequentially and/or simultaneously, so long as the combination is stable until such time that ClO₂ is to be generated. The term “non-reactive,” as use herein, is intended to mean that a component or ingredient as used is not immediately reactive with other components or ingredients present to form chlorine dioxide. The phrase “thickened fluid composition” encompasses compositions which can flow under applied shear stress and which have an apparent viscosity when flowing that is greater than the viscosity of the corresponding aqueous chlorine dioxide solution of the same concentration. This is meant to cover the full spectrum of thickened fluid compositions, including: fluids that exhibit Newtonian flow (where the ratio of shear rate to shear stress is constant and independent of shear stress), thixotropic fluids (which require a minimum yield stress to be overcome prior to flow, and which also exhibit shear thinning with sustained shear), pseudoplastic and plastic fluids (which require a minimum yield stress to be overcome prior to flow), dilantant fluid compositions (which increase in apparent viscosity with increasing shear rate) and other materials which can flow under applied yield stress. The phrase “apparent viscosity” is defined as the ratio of shear stress to shear rate at any set of shear conditions which result in flow. Apparent viscosity is independent of shear stress for Newtonian fluids and varies with shear rate for non-Newtonian fluid compositions.

The chlorine dioxide forming composition comprises a mixture containing a metal chlorite, an acid source, a thickener/thickening component and optionally a free halogen source, wherein these ingredients are combined in a fashion so as to be non-reactive. In one embodiment, at least one of the chlorite, the acid source, and the thickener component is a solid constituent. The solid constituent includes particulates, a unitary solid body or a combination of both. Thus, the mixture may, for example, contain a particulate solid metal chlorite, optionally a particulate solid acid source, optionally a particulate solid free halogen source, and optionally one or more particulate solid thickener agents. One or more of these components can be in water. In another embodiment, the mixture is a solid body comprising a metal chlorite, an acid source and optionally a free halogen source, with particulate solid thickener agent(s). In yet another embodiment, the mixture may be particulates in the form of a powder (for example by grinding) and mixed in a layer of thickener component thereby forming a thickened matrix. The thickened matrix is then disposed on a dental strip which is then adhered to a malleable wax for use on teeth.

The mixture generates a thickened aqueous chlorine dioxide when added to an aqueous medium. The aqueous medium comprises water alone or water with additional components such as either an acid source or a source of chlorite anion (but not both together), one or more thickener agents, and a free halogen source when the aqueous medium does not contain a source of chlorite anions.

The term “particulate” is defined to mean all solid materials. The particulates are interspersed with each other to contact one another in some way. These solid materials include particles comprising big particles, small particles or a combination of both big and small particles.

The metal chlorite employed in the present invention can generally be any metal chlorite. Preferred metal chlorites are alkali metal chlorites, such as sodium chlorite and potassium chlorite. Alkaline earth metal chlorites can also be employed. Examples of alkaline earth metal chlorites include barium chlorite, calcium chlorite, and magnesium chlorite. The most preferred metal chlorite is sodium chlorite.

The acid source may include inorganic acid salts, salts comprising the anions of strong acids and cations of weak bases, acids that can liberate protons into solution when contacted with water, organic acids, and mixtures thereof. The acid source in particular applications of the present invention, is preferably a particulate solid material which does not react substantially with the metal chlorite during dry storage, however, does react with the metal chlorite to form chlorine dioxide when in the presence of the aqueous medium. As used herein the term “acid source” shall mean a particulate solid material which is itself acidic or produces an acidic environment when in contact with liquid and metal chlorite. The acid source may be water soluble or substantially insoluble in water. The preferred acid sources are those which produce a pH of below about 7, more preferably below about 5.

Examples of preferred substantially water soluble acid source forming components include, but are not limited to, water soluble solid acids such as boric acid, citric acid, tartaric acid, water soluble organic acid anhydrides such as maleic anhydride, and water soluble acid salts such as calcium chloride, magnesium chloride, magnesium nitrate, lithium chloride, magnesium sulfate, aluminum sulfate, sodium acid sulfate (NaHSO₄), sodium dihydrogen phosphate (NaH₂PO₄), potassium acid sulfate (KHSO₄), potassium dihydrogen phosphate (KH₂PO₄), and mixtures thereof. The most preferred acid source forming component is sodium acid sulfate (sodium bisulfate). Additional water soluble acid source forming components will be known to those skilled in the art and are included within the scope of the present invention.

As used herein, the term “source of free halogen” or “free halogen source” means a compound or mixtures of compounds which release halogen upon reaction with water. As used herein, the term “free halogen” means halogen as released by a free halogen source. In one embodiment the free halogen source is a free chlorine source and the free halogen is free chlorine. Suitable examples of free halogen source used in the anhydrous compositions include dichloroisocyanuric acid and salts thereof such as sodium dichloroisocyanurate and/or the dihydrate thereof (alternatively referred to as the sodium salt of dichloroisocyanuric acid and/or the dihydrate thereof and hereinafter collectively referred to as “NaDCCA”), trichlorocyanuric acid, salts of hypochlorous acid such as sodium, potassium and calcium hypochlorite, bromochlorodimethylhydantoin, dibromodimethylhydantoin and the like. The preferred source of free halogen is NaDCCA.

The chlorine dioxide forming composition is such that when it is added to liquid water, it will produce a thickened solution of chlorine dioxide and, if a source of free halogen is present, free halogen. In one embodiment, if free halogen is present, the concentration of free halogen, in particular free chlorine, in the solution being:

(a) less than the concentration of chlorine dioxide in the solution on a weight basis and the ratio of the concentration of chlorine dioxide to the sum of the concentrations of chlorine dioxide and chlorite anion in the solution is at least 0.25:1 by weight; or

(b) equal to or greater than the concentration of chlorine dioxide in the solution on a weight basis and the ratio of the concentration of chlorine dioxide to the sum of the concentrations of chlorine dioxide and chlorite anion in the solution is at least 0.50:1 by weight.

Suitable thickeners for producing thickened and pseudo plastic aqueous fluid mixtures of chlorine dioxide include clays, polymers, gums, etc. The thickeners may be in particulate form or may take form as an aqueous medium. Examples of polymers include super absorbent polymers and polyacrylate polymers. Laponite clays, attapulgite clays, bentonite clays are suitable clays and exemplary gums include xanthan and guar gums.

In this invention, the mixture remains stable for some period of the time. The stability is attributed to keeping the mixture anhydrous and/or by using stabilizing components.

The stabilizing components that may be used in the present invention to inhibit premature reaction of the mixture with each other are coatings or encapsulating materials disposed over one or more of the particulate constituents of the invention. These stabilizing components are designed to be slowly, and not immediately, soluble. Preferred coatings or encapsulating materials include, e.g., oleophilic materials and, more preferably, hydrophobic (water-insoluble) polymeric materials. Other non-limiting examples of encapsulating or coating materials which can function as stabilizing component include conventional edible gums, resins, waxes and mineral oils. Such stabilizing coating materials prevent immediate reactions between the mixture and the aqueous medium. The stabilized components may be activated for immediate reaction by techniques known to those of ordinary skill in the art such as breaking the components or removing the stabilizing components to expose the component to aqueous medium by, for example, stirring and heating.

The term “hydrophobic” or “water-insoluble” as employed herein with respect to organic polymers refers to an organic polymer which has a water solubility of less than about one gram per 100 grams of water at 25° C.

Non-limiting examples of suitable water-insoluble polymers, alone or in combination with one or more other components, used herein include polyvinyl acetate, polyacrylamide, polyvinyl chloride, polystyrene, polyethylene, polyurethane, and the like.

Non-limiting examples of suitable oleophilic coatings or encapsulating materials include paraffin, mineral oil, edible oils such as peanut oil, coconut oil, palm oil, or safflower oil, oleophilic organic esters such as isopropyl silomane myristate or isopropyl palmitate, edible polysiloxanes, and the like.

Encapsulating materials containing a mixture of paraffin and waxes are also suitable stabilizing components.

The stabilizing component may stabilize one or more of the components of the mixture. In one instance, at least one of the components is aqueous and other two are stabilized.

When using the solid body form of the anhydrous composition to produce the chlorine dioxide in water of the present invention, the particulate solid components are collectively disposed in a body, such as a unitary body, and then added to the aqueous medium. Solid bodies are discussed in commonly assigned U.S. Pat. Nos. 6,432,322 and 6,699,404 and are incorporated herein by reference. Thus, one method of forming the thickened ClO₂ mixture involves combining the solid body with particulate solid thickeners, or thickener component(s)/agent(s), and then adding both to water. Here, the ClO₂ is produced by the solid body and the mixture is thickened by the thickener agents to produce the final ClO₂ thickened mixture. In an alternative method, the thickener may be incorporated directly into the solid body.

Solid bodies comprise a metal chlorite such as sodium chlorite, an acid source such as sodium bisulfate, optionally a source of free halogen such as the sodium salt of dichloroisocyanuric acid or a hydrate thereof, and optionally a thickener. Preferably the solid body contains less than about 1% wt. free moisture, which can be evolved at 100 degrees Celsius. The solid body is suitable for producing an aqueous solution of chlorine dioxide when immersed in water and thickened chlorine dioxide when a thickener is incorporated directly into the solid body or added as a component separate from the solid body. However, similar to the individual particulates listed above, not all of the constituents of the solid body are immediately soluble in water.

As used herein, the term “solid body” means a solid shape, preferably a porous solid shape, or a tablet comprising a mixture of granular particulate ingredients wherein the size of the particulate ingredients is substantially smaller than the size of the solid body. Such solid bodies may be formed by a variety of means known in the art, such as tableting, briquetting, extrusion, sintering, granulating and the like. The preferred method of forming such solid bodies is by compression, also known as tableting. For reasons of convenience, hereinafter references to tablets and tableting shall be understood to be representative of solid bodies made by any method.

In producing the solid bodies, the metal chlorite comprises an alkali or alkaline earth metal chlorite, preferably sodium chlorite, and most preferably technical grade sodium chlorite comprising nominally 80% by weight sodium chlorite and 20% by weight stabilizing salts such as sodium hydroxide, sodium carbonate, sodium chloride, sodium nitrate and/or sodium sulfate. Suitable acid sources include inorganic acid salts, such as sodium acid sulfate (sodium bisulfate), potassium acid sulfate, sodium dihydrogen phosphate, and potassium dihydrogen phosphate; salts comprising the anions of strong acids and cations of weak bases, such as aluminum chloride, aluminum nitrate, cerium nitrate, and iron sulfate; acids that can liberate protons into solution when contacted with water, for example, a mixture of the acid ion exchanged form of molecular sieve ETS-10 (see U.S. Pat. No. 4,853,202) and sodium chloride; organic acids, such as citric acid and tartaric acid; and mixtures thereof. Preferably, the acid source is an inorganic acid source, and most preferably is sodium bisulfate.

The pore size and pore volume ranges required to facilitate the desired degree of conversion of chlorite anion to chlorine dioxide will depend upon many factors, e.g., the particular combination of reagents in the tablet, the size of the tablet, the shape of the tablet, the temperature of the water, other chemicals dissolved in the water, the desired degree of conversion of chlorite anion to chlorine dioxide, the desired amount of free halogen to be delivered into the solution, etc. Accordingly, it is not believed that there is a single optimum range of pore sizes or pore volumes that will produce an optimum result.

It is within the capability of one skilled in the art to vary the pore size and the pore volume of a tablet to achieve the desired result in respect to the characteristics of the chlorine dioxide solution. For example, the pore size and pore volume may be varied by varying the particle size of the powder used to prepare the tablet or by varying the compaction force used to form the tablet or by varying both the particle size and the compaction force. Larger particles of powder will generally produce larger pores and more pores in the tablet. Increasing compaction force will generally reduce both the size and volume of the pores in the tablet.

The tablets of one embodiment of the invention have been observed to rapidly produce a highly converted solution of free molecular chlorine dioxide, meaning that the conversion ratio (chlorite anion to chlorine dioxide) is 0.25 or above. Preferably, the conversion ratio is at least 0.50, more preferably at least 0.60, and most preferably at least 0.75. The term “conversion ratio” used herein means the calculated ratio of the free chlorine dioxide concentration in the product solution to the sum of free chlorine dioxide plus chlorite ion concentrations in the product solution. Further, the chlorine dioxide solution is rapidly produced in a safe and controlled manner; and when the chlorine dioxide concentration so produced is at typical use levels (about 0.1 to about 1,000 ppm, preferably about 0.5 to about 200 ppm, by weight) in typical tap water, the solution will contain substantially no free chlorine or other free halogen and will have a generally neutral pH.

The term “rapidly produced” as used herein means that total chlorine dioxide production is obtained in less than about 8 hours, preferably in less than about 2 hours and most preferably in less than about 1 hour. The term “no free chlorine or other free halogen” used herein means that the concentration of free chlorine or other free halogen in solution is less than the concentration of chlorine dioxide in said solution on a weight basis, preferably less than ½ the concentration of chlorine dioxide in said solution, more preferably less than ¼ the concentration of chlorine dioxide, and most preferably no more than 1/10 the concentration of chlorine dioxide, on a weight basis.

The term “generally neutral pH” used herein means that the pH is higher than that normally required to form substantial concentrations of chlorine dioxide in solution (i.e., pH higher than about 2) and lower than the pH at which chlorine dioxide is known to disproportionate in solution (i.e., pH below about 12). Preferably, the pH of the resultant solution is between about 4 and 9 to minimize the potential for corrosion of materials with which the solution comes into contact. More preferably the pH of the resultant solution should be in the range of about 5-9, and most preferably in the range of about 6-9; ideally the pH will be 7. In certain cases, it may be advantageous to produce chlorine dioxide in a solution that is already at either a higher or a lower pH than the pH of about 7. The solid bodies may be used to deliver chlorine dioxide into such solutions without materially changing the pH of the solution when the chlorine dioxide concentration is at typical use levels. For example, if a solid body is used to produce chlorine dioxide in a typical solution of laundry detergent, it is advantageous for the detergent solution to be at alkaline pH (i.e., >9) where the detergent functions best. The solid body may be used for that purpose. In such cases, however, it is preferred that the pH of the resultant detergent/chlorine dioxide solution be below about 12, as chlorine dioxide degrades at a pH higher than about 12.

It is often advantageous for the free halogen concentration of the resultant solution to be low, as free halogen can lead to corrosion of materials in which the solution comes into contact, and free halogen can react with organic materials to produce toxic halogenated hydrocarbons. Because of the ability of the solid body to produce highly converted solutions of chlorine dioxide in the absence of a free halogen source, it is possible to use sufficiently low amounts of a free halogen source in the solid body tablet formulation to accelerate the chlorine dioxide formation reaction without contributing excessive amounts of free halogen to the resultant solution.

In other situations, the presence of a relatively high concentration of free chlorine or other free halogen in solution may be acceptable. In such situations, it is possible to use the solid bodies to produce very highly converted aqueous solutions of chlorine dioxide where the ratio of the concentration of chlorine dioxide in solution to the sum of the concentrations of chlorine dioxide and chlorite anion is greater than 0.5 on a weight basis. In those cases, the concentration of free chlorine or other free halogen in solution may be equal to or even greater than the concentration of chlorine dioxide in solution on a weight basis.

The tablets may, if desired, contain additional optional ingredients, that may be useful, for example, to assist in the tableting process, to improve the physical or aesthetic characteristics of the produced tablets and to assist tablet solubilization and/or the yield of chlorine dioxide obtained. Such ingredients include but are not limited to fillers such as attapulgite clay and sodium chloride; tableting and tablet die lubricants; stabilizers; dyes; anti-caking agents; desiccating agents such as calcium chloride and magnesium chloride; pore forming agents such as a swelling inorganic clay, e.g., Laponite clay available from Southern Clay Products, Inc., and a framework former that can react with one or more other constituents in the formulation to produce a low solubility porous framework structure in which the chlorine dioxide forming reactions may proceed.

Effervescing agents such as sodium bicarbonate may be included in small amounts, e.g., about 1 to about 50 wt. %, based on the weight of the solid body, but these effervescing agents can reduce the conversion of chlorite anion to chlorine dioxide by accelerating breakup and dissolution of the tablet.

Two general types of tablet devices are included in the tablet embodiment of the present invention. One type of device comprises tablets that are fully soluble in water, and the preferred formulation of such tablets comprises dried powdered technical grade sodium chlorite, a dried powdered acid source, preferably sodium bisulfate and a non-reactive thickener. As mentioned above, the thickeners may be incorporated directly into the solid body or added as a component separate from the solid body.

Additional dried powdered ingredients such as magnesium chloride may optionally be added to even further improve the yield and rate of production of the chlorine dioxide. The dried powdered ingredients are mixed and the resultant powdered mixture is compressed in a tablet die at a force sufficient to produce a substantially intact tablet, typically about 1000-10,000 lb./in.².

The resultant tablets are stable during storage as long as the tablets are protected from exposure to water (either liquid or vapor). The tablets rapidly produce a highly converted solution of free chlorine dioxide when immersed in water.

The second type of device comprises tablets that are not fully soluble in water at a high rate. These non-fully soluble tablets are designed to have (or produce) a low solubility or slowly soluble porous framework structure in which the chlorine dioxide forming reactions may proceed to substantial completion prior to dissolution of the porous framework. Generally tablets of this second type convert a greater proportion of their chlorite anion precursor chemical to chlorine dioxide compared to the fully soluble tablets described above.

The preferred formulation for this second type of tablet device comprises particulate powdered sodium chlorite, particulate powdered sodium bisulfate, particulate powdered calcium chloride and a non-reactive thickener. A particulate powdered clay such as Laponite clay may optionally be added to even further improve the yield and rate of production of the chlorine dioxide. Here, the Laponite clay that is optionally incorporated directly into the solid body cannot be used as a thickener for forming the thickened chlorine dioxide solution. When utilized in the tablets, the Laponite clay is trapped in the pores of the low solubility or slowly soluble porous framework of the second tablet and is not released into the bulk solution which would allow the clays to aggregate and form a viscous medium. Laponite clay may still be used to form the thickened chlorine dioxide solution by adding the clay as a separate component with the solid body to water. In these second tablet types, the polymers or gums maybe also used as thickeners to form the thickened chlorine dioxide solution. The polymers or gums, unlike the Laponite clay, can be directly added to the tablet of the second type or, alternatively, the gums or polymers can be added as a separate component along with the solid body to water.

As with tablets of the first type, the particulate powdered ingredients are mixed and the resultant powdered mixture is compressed in a tablet die at a force sufficient to produce a substantially intact tablet, typically about 1000-10,000 lb./in.². The resultant tablets are stable during storage as long as the tablets are protected from exposure to water (either liquid or vapor). When immersed in water, the tablets rapidly produce a highly converted solution of free chlorine dioxide.

Tablets of this second type generally provide more efficient conversion of chlorite anion to chlorine dioxide compared to tablets of the first type. It is believed that this occurs because the low solubility porous framework provides a favorable environment for the chlorine dioxide forming reactions to proceed until substantial exhaustion of the reactants.

Chlorine dioxide formation in tablets of the second type of device is believed to occur substantially within the favorable environment of the pore space of the low solubility (or slowly soluble) porous framework and is simultaneously thickened with the thickeners. Since the favorable pore structure of this framework appears to remain substantially intact during this reaction time, substantially all of the chlorite anion has an opportunity to react and form chlorine dioxide under favorable conditions within the pores. This maximizes chlorite conversion to chlorine dioxide. In contrast, a device of the first type is being dissolved into the bulk solution at the same time that it is producing chlorine dioxide. Since it is believed that the reagents will only react at a practically useful rate under concentrated conditions (such as those that exist within the pores of the tablets), that fraction of the chlorite that dissolves into bulk solution prior to conversion to chlorine dioxide will substantially remain as chlorite and not be converted to chlorine dioxide under the generally dilute conditions of the bulk solution.

The low solubility porous framework of the preferred composition of the second type of tablet device comprises a framework former such as a low solubility compound such as calcium sulfate, calcium phosphate, aluminum phosphate, magnesium phosphate, ferric sulfate, ferric phosphate or zinc phosphate; or a low solubility amorphous material such as silica-alumina gel, silica-magnesia gel, silica-zirconia gel, or silica gel; and may additionally include a clay or other substantially insoluble framework or pore former such as Laponite clay. The calcium sulfate preferably is formed from the reaction between calcium cations e.g., from the calcium chloride constituent and sulfate anions derived from the sodium bisulfate constituent. Other sources of calcium cations such as calcium nitrate as well as other sources of sulfate anions such as magnesium sulfate may also be used. Phosphate anion preferably is provided by use of soluble phosphate compounds such as sodium phosphate, sodium hydrogen phosphate, sodium dihydrogen phosphate, the corresponding potassium phosphate salts, as well as other soluble phosphate salts. The silica alumina gel preferably is formed from the reaction between sodium silicate and aluminum sulfate. Silica-magnesia gel preferably is formed from the reaction between sodium silicate and magnesium sulfate. Silica-zirconia gel preferably is formed from the reaction between sodium silicate and zirconyl sulfate. Silica gel preferably is formed from the reaction between sodium silicate and acidity from the solid acid source. Additional solid acid component may be required to compensate for the alkalinity of the sodium silicate constituent.

The preferred clay, Laponite clay, is insoluble as provided and is not released into the bulk solution. It is a swelling clay that become trapped within the pores, and enhances the pore structure of the porous framework by forming cracks and cavities as it swells. As stated previously, the Laponite clay is trapped in the low solubility or slowly soluble porous framework structure of the second tablet and thus does not escape into the surrounding water to form a viscous medium. We have found that forming the low solubility porous framework, e.g., the calcium sulfate, calcium phosphate, aluminum phosphate, etc., frameworks in-situ via chemical reaction is particularly advantageous and that the chlorine dioxide yield from tablets wherein the framework is formed in-situ is significantly better (nominally 25% better) than tablets in which the framework material is a constituent of the initial powder formulation. The presence of the clay in addition to the framework material provides only a small improvement over the use of the framework material, without the clay.

The term “low solubility or slowly soluble porous framework” used herein means a porous solid structure that remains substantially undissolved in the product solution during the period of chlorine dioxide production. It is not necessary that the porous framework remain wholly intact during the reaction time to form chlorine dioxide. One aspect of this invention includes tablets of the second type in which the tablet disintegrates into substantially insoluble (or slowly soluble) granules that release chlorine dioxide into solution. This is acceptable, we believe, because the size of the granules is still large relative to the size of the pores within the pore space of the granules, so the necessary concentrated reaction conditions exist within the pore space despite the breakdown of the framework into granules. Typically, the framework former will be present in an amount of about 10 to about 90 wt. %, based on the weight of the solid body.

In tablet devices of both types, it is preferred that the powdered ingredients be dry prior to mixing and tableting in order to minimize premature chemical interaction among the tablet ingredients.

General Procedures for Making and Testing the Tablets of the Invention Tablet Formation:

The individual chemical components of the tablet formulation are dried prior to use. The desired amount of each component is carefully weighed into a plastic vial. In the following examples, formulations are given on a weight percent basis. The vial containing all the components of the tablet formulation is shaken to mix the components thoroughly. The contents of the vial are emptied into an appropriately sized die (e.g., a 13-mm diameter for a 1 g tablet). The plunger is placed in the die and the contents are pressed into a pellet using a hydraulic laboratory press. The maximum force reading on the press gauge was 2000 pounds unless otherwise noted. This force on the tablet punch may be converted to pounds/in.² if the area of the face of the plunger in in.² is known (typically 0.206 in.² for a 1 g tablet). The resulting tablet is removed from the die and placed in a closed plastic vial until use (typically within 10 minutes).

Tablet Performance:

The tablet is placed in a volumetric flask or container filled with a known amount of tap water. Chlorine dioxide evolution starts immediately as evidenced by bubbles and the appearance of a yellow color. The tablet is allowed to react until completion. Completion of the reaction depends, in part, on the tablet type and size. Typically the reaction time is 2 hours or less if a 1 g tablet is partially insoluble and 0.5 hr. if a 1 g tablet is completely soluble. When reaction is complete, the flask/container is shaken or stirred in order to mix the contents. Then the contents are analyzed. Typically, chlorine dioxide is measured by UV-Vis spectrometry, using four wavelengths (the average value is reported). Chlorite and chlorine are measured by titration of typically 25 ml of chlorine dioxide solution using procedures equivalent to those found in the text, Standard Methods for the Examination of Water and Wastewater, 19^{th} Edition (1995) pages 4-57 and 4-58. This text is published jointly by the American Public Health Association, The American Water Works Association and the Water Environment Federation. The publication office is American Public Health Association, Washington, D.C. 20005. Total oxidants are measured by titration using a Brinkmann Autotitration System, 716 DMS Titrino equipped with a massive platinum electrode (Brinkmann Part No. 6.0415.100). The method is an iodimetric titration in an acid medium based on the oxidation of iodide to iodine and its subsequent reaction with the titrant, sodium thiosulfate. The typical procedure was as follows. One hundred milliliters of chlorine dioxide solution and a stirring bar were placed in a beaker and 2 g of potassium iodide (Reagent Crystals) and 10 ml of a 1N solution of sulfuric acid (Mallinckrodt) were added with stirring. The resulting solution is titrated with 0.1 N thiosulfate solution (Aldrich Chemical Co.). The endpoint is automatically determined by the Brinkmann Titrino software. This endpoint is used to calculate the concentration of total oxidants in the sample. The pH of the original chlorine dioxide solution is measured using a pH electrode either on the solution “as is” and/or diluted with sufficient water to give approximately a 10 ppm concentration of chlorine dioxide.

Another method for producing thickened mixtures having a high concentration of ClO₂ (>10 ppm), includes providing as the particulate constituent particulate solid sodium chlorite and particulate solid thickener agent(s) and then combining the particulates with an acidic aqueous solution having sufficient excess acidity. The pH of the resultant mixture is <4 subsequent to the addition of the particulate constituent. Still another alternative would comprise a particulate solid acid source as the particulate constituent and a sodium chlorite solution containing one or more thickener agents as the aqueous medium. The pH of the resultant mixture after mixing of the particulate and is <4. Other variations are also within the scope of this invention.

The thickened fluid composition comprising chlorine dioxide may be made instantaneously by combining the mixture with the aqueous medium. Alternatively, the mixture and the aqueous medium may be retained in a dispensing unit that separates the mixture from the aqueous medium immediately prior to use, and allows the two constituents to combine when dispensed.

The dispensing unit can comprise a single housing unit having a separator or divider integrated with the housing so the mixture and the aqueous medium only meet after being dispensed from the dispensing unit. Alternatively the dispensing unit can comprise a single housing unit having a frangible separator or divider that initially separates the mixture and aqueous medium, but then permits the mixture and aqueous medium to mix when the frangible divider is penetrated. Still another variation on the dispensing unit involves a dispensing unit that holds at least two individual frangible containers, one for the mixture constituents and the other for the aqueous medium; the individual frangible containers break upon the application of pressure. These and other dispensing units are fully described in U.S. Pat. No. 4,330,531 and are incorporated herein by reference.

Chlorine dioxide has established uses in bleaching textiles and pulp in making paper, deodorizing, disinfecting, sanitizing and sterilizing surfaces or spaces. The present invention can further be used in wound dressings, environmental cleanup, dental/oral care substances, germ killing material, tooth whitening compositions, and personal lubricants among a variety of other applications

Other uses include oxidizing foul smelling compounds; treating cooling towers, emergency drinking water, car wash recycle water, water softeners as well as animal confinement facilities; and sanitizing hard, nonporous food contact surfaces and utensils. The present invention can also be used in typical industrial applications such as in food processing plants, breweries, and food handling establishments, recirculating cooling water systems and in general water treatment facilities.

When the composition is used as a therapeutic membrane such as a wound dressing it may further include a fluid polymerizable composition comprised of polymerizable organic compounds and photoinitiators. U.S. Pat. No. 5,597,561, discloses an example of a thickened wound dressing. The '561 patent is directed to an adherent disinfecting composition which includes metal chlorites and other ingredients in the composition. The '561 composition provides an effective adherent matrix that acts as a disinfectant barrier for preventing transmission and propagation of microbial infections.

When the composition is utilized in tooth whitening compositions, the composition may be disposed in a dental tray wherein the composition can be placed against the tooth surface via the tray. The composition remains in contact with the tooth surface for a predetermined period of time. The tooth surface is whitened through the oxidative action of chlorine dioxide on chromaphores entrapped within the acquired pellicle, enamel, and dentin structures of the tooth. Though not required, the tooth whitening composition may have select flavorants and sweeteners incorporated into the composition. Alternatively, the composition may be utilized in tooth whitening compositions via a dental strip or a monolithic sheath. The sheath is a matrix comprised of particulate chlorine dioxide forming components disposed in a thickener such as a super absorbent polymer. The matrix or sheath may be shaped in the form of a strip so that it can be handled and applied directly to the teeth or adhered to a strip of malleable wax or other sheet material for application on the teeth. The thickened chlorine dioxide mixture forms on the sheath upon contact with water or other aqueous medium.

In order to demonstrate the invention, some examples are set forth below.

### Example 1

A 250 mg tablet of the composition described in Example 5 of U.S. Pat. No. 6,699,404, was combined with 0.3 grams of ASAP 2000, a sodium acrylate super-absorbent polymer powder supplied by Chemdal Corporation of Palatine, Ill. The above mixture was combined with 20 ml of tap water in a clear glass vial and gently shaken and stored overnight to produce a thick aqueous mixture comprising chlorine dioxide (ClO₂.) The mixture was a thickened, yet fluid composition. See Table 1.

### Example 2

The procedure of Example 1 was repeated with 0.4 grams of ASAP 2000 acrylate powder. The mixture was a thickened, yet fluid composition. See Table 1.

### Example 3

The procedure of Example 1 was repeated with 0.5 grams of ASAP 2000 acrylate powder. The mixture was a plastic, thickened composition that flowed when inverted. See Table 1.

### Example 4

The procedure of Example 1 was repeated with 0.6 grams of ASAP 2000 acrylate powder. The mixture was a plastic, thickened composition that flowed when inverted. See Table 1.

### Example 5

The procedure of Example 1 was repeated with 0.7 grams of ASAP 2000 acrylate powder. The mixture was a plastic, thickened composition that did not flow when inverted. See Table 1.

TABLE 1

| | | | | Exam- | Exam- |
|---|---|---|---|---|---|
| | Example 1 | Example 2 | Example 3 | ple 4 | ple 5 |
| Grams of | 0.3 | 0.4 | 0.5 | 0.6 | 0.7 |
| ASAP 2000 | | | | | |
| Mixture | Thickened, | Thickened, | Plastic, | Plastic, | Plastic, |
| consistency | still fluid | still fluid | but flowed | but | and did |
| result | | | when | flowed | not flow |
| | | | inverted | when | when |
| | | | | inverted | inverted |

Table 1 indicates the grams of the ASAP used in each example and indicates the resulting mixture consistency for Examples 1-5. Table 1 shows that the amount of ASAP is related to the thickened consistency of the mixture.

### Example 6

A 250 mg tablet of the composition described in Example 5 of U.S. Pat. No. 6,699,404, was immersed in 20 ml of tap water in a clear glass vial and allowed to react without stirring until dissolved. The solution was then divided into two equal parts and 3.5 grams of ASAP 2000 acrylate powder was added to one of the portions (with stirring.) Each portion was diluted with 100 ml using tap water.

The unthickened portion was analyzed for ClO₂ concentration by UV/Visible spectroscopy using a Spectral Instruments Model 440 UV/Visible spectrometer with a direct insertion probe. Both diluted solutions were analyzed for free oxidant concentration by KI/thiosulfate titration buffered at a pH of 7. The results showed that the unthickened solution contained about 900 ppm ClO₂ (902 ppm ClO₂ by UV/Visible spectroscopy and 875 by titration.) The thickened mixture contained 821 ppm ClO₂ by titration. Based on this result it was concluded that ClO₂ could be stable in a thickened aqueous mixture comprising an organic thickening agent.

### Example 7

The test of Example 6 was repeated and the unthickened solution contained 1100 ppm ClO₂ (1170 ppm by UV/Visible spectroscopy and 1062 ppm by titration.) The thickened mixture contained 991 ppm ClO₂ by titration. Based on this result it was concluded that ClO₂ could be stable in a thickened aqueous mixture comprising an organic thickening agent.

### Example 8

Ten tablets of the composition described in Example 5 of U.S. Pat. No. 6,699,404, were dissolved in 200 ml of tap water to produce a solution of chlorine dioxide. To each of seven clear glass vials was added 0.7 grams of ASAP 2000 acrylate powder followed by 20 mls of ClO₂ solution prepared above. Each vial was gently shaken until a gel formed. The ClO₂ concentration of one vial was measured immediately by titration and found to be 766 ppm. The remainder were tightly capped and stored in the dark at ambient lab temperature and humidity. At selected time intervals a vial was removed from storage and analyzed to determine the residual ClO₂ concentration. See Table 2 below.

TABLE 2

| Day | Result |
|---|---|
| 1 | 648 ppm |
| 4 | 454 ppm |
| 6 | 522 ppm |
| 20 | 330 ppm |
| 49 | 530 ppm |

This demonstrated the surprisingly good stability of ClO₂ in the thickened mixture. About 25% of the ClO₂ was lost from the solution within a week, and the concentration was substantially unchanged thereafter.

### Example 9

Thickened chlorine dioxide provides a way to give off controlled release of chlorine dioxide into air. The chemical stability of chlorine dioxide in thickened mixtures can be affected by the type of thickener used in the composition. Some thickeners reduce the chemical stability of chlorine dioxide. The chemical stability of chlorine dioxide in thickened mixtures was measured using different thickeners including Laponite clay, xanthan gum, guar gum, and Polyox™ brand polyethylene oxide. The chemical stability was tested at both 0.1% and 1% by weight thickener concentrations. Table 3 below indicates the retention of chlorine dioxide concentration of the thickened chlorine dioxide solution (%) after twenty minutes.

TABLE 3

| | ClO2 concentration |
|---|---|
| Thickener | retention (%) |
| Laponite clay 1% | 92 |
| Laponite clay 0.1% | 92 |
| Guar gum 1% | 30 |
| Guar gum 0.1% | 57 |
| Xanthan 1% | 67 |
| Xanthan 0.1% | 70 |
| Polyox ™ 1% | 74 |
| Polyox ™ 0.1% | 81 |
| Water Control | 91 |

The data shows that the chlorine dioxide is more stable in some thickeners than others. Here, at the twenty minute mark, the concentration of chlorine dioxide was weakest in both 0.1% and 1% by weight guar gum concentrations, and highest in both 0.1% and 1% by weight Laponite clay concentrations.

What is claimed is:

1. A stable composition for generating in an aqueous medium a thickened fluid containing chlorine dioxide comprising:
a mixture containing a chlorite, an acid source and a thickener component, wherein said components are combined so as to be non-reactive and to produce a thickened aqueous chlorine dioxide when the acid source reacts with the chlorite to form chlorine dioxide in the presence of an aqueous medium.

2. The composition of claim 1 consisting essentially of:
a mixture containing a chlorite, an acid source and a thickener component, and optionally one or more of: a free halogen source; a coating or encapsulating material selected from an oleophilic material and a hydrophobic polymeric material; a filler; a die lubricant; a stabilizer; a dye; an anti-caking agent; a dessicating agent; a pore forming agent; and a framework former;
wherein said components are combined so as to be non-reactive and to produce a thickened aqueous chlorine dioxide when the acid source reacts with the chlorite to form chlorine dioxide in the presence of an aqueous medium.

3. The composition of claim 1, wherein said components are dry solid particulates.

4. The composition of claim 1, wherein said mixture comprises a unitary solid body comprising at least two of said chlorite, said acid source and said thickener component.

5. The composition of claim 4, wherein the solid body comprises said chlorite, said acid source, optionally said thickener component, and optionally a free halogen source, wherein said chlorite is a metal chlorite.

6. The composition of claim 4, wherein the solid body comprises said chlorite, said acid source, said thickener component, and optionally a free halogen source, wherein said chlorite is a metal chlorite and said thickener component is non-reactive.

7. The composition of claim 1, wherein said mixture is a combination of said thickener component and a unitary solid body comprising said chlorite and said acid source, wherein said thickener is particulate.

8. The composition of claim 1, wherein the thickener is selected from clays, polymers and gums.

9. The composition of claim 1, wherein the at least one of said chlorite or said acid source is in an aqueous medium and at least one of said chlorite or said acid source is treated with a stabilizing component.

10. The composition of claim 1, wherein the mixture is anhydrous containing less than about 1% wt. free moisture.

11. A thickened aqueous chlorine dioxide fluid composition comprising: the composition of claim 1 and an aqueous medium.

12. A device for use on teeth, said device comprising a composition for generating a thickened fluid containing chlorine dioxide, the composition consisting essentially of:
a mixture containing a chlorite, an acid source and a thickener component, and optionally one or more of: a free halogen source; a coating or encapsulating material selected from an oleophilic material and a hydrophobic polymeric material; a filler; a die lubricant; a stabilizer; a dye; an anti-caking agent; a dessicating agent; a pore forming agent; and a framework former;
wherein said components are combined so as to be non-reactive and to produce a thickened aqueous chlorine dioxide when the acid source reacts with the chlorite to form chlorine dioxide in the presence of an aqueous medium.

13. The device of claim 12, wherein said device is selected from the group consisting of a dental tray, a dental strip and a monolithic sheath.

14. The device of claim 13, wherein said device is a monolithic sheath and said sheath is a matrix, wherein said matrix comprises said composition.

15. A wound dressing comprising a composition for generating a thickened fluid containing chlorine dioxide, the composition consisting essentially of:
a mixture containing a chlorite, an acid source and a thickener component, and optionally one or more of: a free halogen source; a coating or encapsulating material selected from an oleophilic material and a hydrophobic polymeric material; a filler; a die lubricant; a stabilizer; a dye; an anti-caking agent; a dessicating agent; a pore forming agent; a framework former; and a fluid polymerizable composition;
wherein said components are combined so as to be non-reactive and to produce a thickened aqueous chlorine dioxide when the acid source reacts with the chlorite to form chlorine dioxide in the presence of an aqueous medium.

16. The wound dressing of claim 15, further comprising a fluid polymerizable composition.
